(19) United States
(12) Patent Application Publication
(10) Pub. No.: US 2003/0120102 A1
(43) Pub. Date: Jun. 26, 2003

(54) Method for the preparation of (s) -2-acetylthio-3-phenylpropionic acid

(76) Inventors: Franciscus Alphons Marie Lommen, Horst (NL); Helmut Koller, Regensburg (DE); Herbert Scherubl, Regensburg (DE)

Correspondence Address: Kate H Murashige, Morrison & Foerster, Suite 500, 3811 Valley Center Drive, San Diego, CA, 92130-2332, US

(21) Appl. No.: 10/203,698

(22) PCT Filed: Feb. 2, 2001
(86) PCT No.: PCT/NL01/00078

(30) Foreign Application Priority Data
Feb. 11, 2000 (NL) 1014354

(51) Int. Cl. C07C 327/32

(52) U.S. Cl. 558/252

(57) ABSTRACT

Method for the preparation of (S)-2-acetylthio-3-phenylpropionic acid, wherein (R)-2-bromo-3-phenylpropionic acid is contacted with thioacetic acid and an organic base, for example triethylamine. Preferably the base is metered to a mixture of (R)-2-bromo-3-phenylproprionic acid and thioacetic acid at a temperature between −10° C. and +30° C.

(R)-2-bromo-3-phenylpropionic acid is preferably prepared starting from D-phenylalanine, sodium nitrite, HBr and a bromide salt, in an aqueous solution at a temperature between −10 and 30° C., and subsequently without isolation converted into (S)-2-acetylthio-3-phenylpropionic acid. The (S)-2-acetylthio-3-phenylpropionic acid obtained can be used in the preparation of pharmaceuticals, in particular ACE inhibitor such as Omapatrilat.

The invention relates to a method for the preparation of (S)-2-acetylthio-3-phenylpropionic acid wherein (R)-2-bromo-3-phenylpropionic acid is contacted with thioacetic acid and an organic base.

It is known to carry out similar conversions with the aid of thioacetic acid and an alkali metal carbonate or bicarbonate, for example from M.-C. Fournie-Zaluski et al., Eur. J. Biochem., 139 (1984) 267, and U.S. Pat. No. 5,508,272, or with an alkali metal salt of thioacetic acid, for example from P. Coric et al., J. Med. Chem., 29 (1996) 1210, and WO-A-99/42438.

Surprisingly it has however been found that with the method according to the invention significantly less by-product is obtained and thereby a higher efficiency is achieved.

Suitable examples of an organic base are alkyl amines, in particular trialkyl amines; heterocyclic amines, in particular pyridines; and (alkyl)anilines. Preferably triethyl amine is used.

Preferably, in the preparation of (S)-2-acetylthio-3-phenylpropionic acid from (R)-2-bromo-3-phenylpropionic acid the organic base is metered to a mixture of (R)-2-bromo-3-phenylpropionic acid and thioacetic acid. Another metering sequence is in principle also possible.

The temperature at which this reaction takes place lies preferably between −10 and +30° C., in particular between −5 and +10° C.

The quantity of thioacetic acid to be added lies preferably between 0.8 and 2, in particular between 0.9 and 1.6 equivalent calculated in relation to the total quantity of D-phenylalanine; or between 1 and 2, in particular between 1.1 and 1.7 equivalent calculated in relation to the total quantity of (R)-2-bromo-3-phenylpropionic acid.

The quantity of organic base to be added lies preferably between 0.8 and 2, in particular between 1 and 1.8 equivalent calculated in relation to the total quantity of D-phenylalanine; or between 1 and 2, in particular between 1.2 and 1.8 equivalent calculated in relation to the total quantity of (R)-2-bromo-3-phenylpropionic acid.

After the reaction the organic base and the excess of thioacetic acid can be removed, for example through extraction at a pH between 0 and 4.

(S)-2-acetylthio-3-phenylpropionic acid is a suitable intermediate product in the preparation of pharmaceuticals, for example in the preparation of ACE inhibitors, for example Omapatrilat (known under the commercial name Vanlev), or similar pharmaceuticals.

The starting product (R)-2-bromo-3-phenylpropionic acid can be prepared in the known way from D-phenylalanine with the aid of NaNO₂ and a Br⁻ compound. Preferably this conversion is carried out however in the presence of HBr and of a bromide salt. The resulting (R)-2-bromo-3-phenylpropionic acid can be used if desired without interim isolation in the conversion to (S)-2-acetylthio-3-phenylpropionic acid.

Suitable bromide salts are for example alkali metal or earth alkali metal salts of HBr, for example NaBr, KBr or CaBr₂. As a rule a more than equivalent quantity of Br⁻ (HBr and bromide salt) is used, preferably 3-10 equivalents, more in particular 4-8 equivalents of Br⁻ calculated in relation to the total quantity of D-phenylalanine. Use of larger quantities of Br⁻ is in principle possible, but provides no significant advantage. The quantity of bromide salt is dependent on the desired excess of Br⁻ and lies preferably between 0.5 and 7 equivalents, in particular between 1.5 and 3 equivalents, calculated in relation to the total quantity of D-phenylalanine.

In a particularly suitable embodiment at least a part of the bromide salt is formed in situ from HBr and a base. Suitable bases that can be used for that purpose are for example alkali metal hydroxides, carbonates or bicarbonates. Preferably KOH or NaOH is used as base.

The quantity of base to be used is dependent on the desired excess of Br⁻ and the desired quantity of bromide salt, and lies preferably between 0.5 and 7, in particular between 1.5 and 3 equivalents, calculated in relation to the total quantity of D-phenylalanine.

The temperature at which the conversion of D-phenylalanine into (R)-2-bromo-3-phenylpropionic acid is carried out lies between −10 and 30° C., for instance between −10 and 20° C., preferably between −5 and 20° C., for instance between −5 and 10° C.

The quantity of sodium nitrite to be used lies preferably between 0.8 and 2 equivalents, in particular between 1 and 1.6 equivalents of sodium nitrite calculated in relation to the total quantity of D-phenylalanine.

The preparation of the (R)-2-bromo-3-phenylpropionic acid is carried out preferably in the presence of an organic solvent, for example a hydrocarbon, preferably a (halogenated) aromatic hydrocarbon. Preferably xylene or toluene is used as organic solvent.

The invention will now be elucidated further by means of examples without however being limited thereby.

### EXAMPLE

Preparation of R-2-bromo-3-phenylpropionic Acid

46.0 ml water was supplied to a 1-litre double-walled glass reactor connected to a coolant.

275.5 g HBr 48% was added. Jacket cooling and stirring were started. Subsequently 67.7 gram KOH 45% was slowly added.

The reaction mixture was cooled to 30-40° C.

45.0 g D-phenylalanine was added to the reaction mixture. Subsequently 213 ml toluene was added to the reaction mixture. The reaction mixture was cooled to 3° C.

95.9 g 30% NaNO₂ solution in water was metered into the reaction mixture in 6 hours. The temperature was kept at 5° C. After the reaction stirring was continued for 3 hours at 3° C.

The reaction mixture was heated to 20° C. Stirring was stopped and the aqueous phase was separated off.

Then the toluene phase was additionally extracted two times with 95 ml water.

The reaction mixture was heated to 70° C. and with the aid of a vacuum pump it was brought under a 100 mbar vacuum. Using a Dean-Stark setup the water was distilled off until the toluene phase was water-free.

Yield: 84.0% R-2-bromo-3-phenylpropionic acid in the toluene solution, relative to D-phenylalanine.

Preparation of S-acetylthiophenyl Propionic Acid.

The toluene solution of R-2-bromo-3-phenylpropionic acid prepared from 45.0 g D-phenylalanine was cooled to 0° C. Subsequently 27.0 g thioacetic acid was added.

In 6 hours 38.5 g triethylamine was metered at a temperature of 0° C. to the reaction mixture.

Then the reaction mixture was heated to 10° C. Stirring was continued until the conversion via HPLC was complete.

95 ml water was added to the reaction mixture and the reaction mixture was heated to 20° C.

With the aid of HCl 32% the reaction mixture was brought to pH=3.4. Stirring was stopped and the water was separated off.

Next, the reaction mixture was washed with 95 ml sodium thiosulfate solution (5%).

With the aid of HCl 32% the reaction mixture was brought to pH=0.75. Subsequently the aqueous phase was separated off and the toluene phase once again extracted with 95 ml water.

Using a Dean-Stark set-up the water was distilled off azeotropically at 60° C. and 100 mbar until the toluene phase was water-free.

The toluene phase was boiled down to 150 ml and filtered at a temperature of approx 40° C.

At 40° C. 360 ml boiling point spirit 80-110 was added, followed by cooling to 0° C.

Yield (after crystallization): 410 g ≅67.1% relative to D-phenylalanine.

1. Method for the preparation of (S)-2-acetylthio-3-phenylpropionic acid, wherein (R)-2-bromo-3-phenylpropionic acid is contacted with thioacetic acid and an organic base.

2. Method according to claim 1, wherein an alkylamine, pyridine or a (alkyl)aniline is used as the organic base.

3. Method according to claim 2, wherein triethylamine is used as the organic base.

4. Method according to any one of the claims 1-3, wherein the base is metered to a mixture of (R)-2-bromo-3-phenylpropionic acid and thioacetic acid at a temperature between −10° C. and +30° C.

5. Method according to claim 4, wherein the temperature lies between −5° C. and 10° C.

6. Method according to one of the claims 1-5, wherein the total quantity of thioacetic acid used lies between 1 and 2 equivalents relative to the quantity of (R)-2-bromo-phenyl-propionic acid.

7. Method according to one of the claims 1-6, wherein the total quantity of organic base used lies between 1 and 2 equivalents relative to the total quantity of (R)-2-bromo-3-phenylpoprionic acid.

8. Method according to one of the claims 1-7, wherein first (R)-2-bromo-3-phenylpropionic acid is prepared starting from D-phenylalanine, sodium nitrite, HBr and a bromide salt, in an aqueous solution at a temperature between −10 and 30° C.

9. Method according to claim 8, wherein the total quantity of HBr plus bromide salt lies between 3 and 10 equivalents relative to the quantity of D-phenylalanine.

10. Method according to claim 9, wherein the quantity of HBr plus bromide salt lies between 4 and 8 equivalents relative to D-phenylalanine.

11. Method according to one of the claims 8-10, wherein the quantity of bromide salt lies between 0.5 and 7 equivalents relative to the quantity of D-phenylalanine.

12. Method according to one of the claims 8-11, wherein at least part of the bromide salt is formed in situ from HBr and a base.

13. Method according to claim 12, wherein alkali metal hydroxide, carbonate or bicarbonate is used as base.

14. Method according to claim 13, wherein KOH or NaOH is used as base.

15. Method according to one of the claims 12-14, wherein the total quantity of base uses lies between 0.5 and 7 equivalents relative to the total quantity of D-phenylalanine.

16. Method according to one of the claims 8-15, wherein the temperature lies between −5° C. and +20° C.

17. Method according to one of the claims 8-16, wherein the quantity of sodium nitrite lies between 1 and 1.4 equivalents of sodium nitrite relative to the quantity of D-phenylalanine.

18. Method according to one of the claims 8-17, wherein the reaction is carried out in the presence of an organic solvent.

19. Method according to claim 18, wherein toluene or xylene is used as organic solvent.

20. Method according to one of the claims 1-19, wherein the (S)-2-acetylthio-3-phenylpropionic acid obtained is converted into a pharmaceutical product, in particular an ACE inhibitor, for example Omapatrilat.
